(19) United States
(12) Patent Application Publication
(10) Pub. No.: US 2016/0296450 A1
(43) Pub. Date: Oct. 13, 2016

(54) Composition Comprising Saccharides and Bicarbonate

(71) Applicant: Amy Ellen Myers, Minneapolis, MN (US)

(72) Inventor: Amy Ellen Myers, Minneapolis, MN (US)

(21) Appl. No.: 15/089,405

(22) Filed: Apr. 1, 2016

Related U.S. Application Data
(60) Provisional application No. 62/142,050, filed on Apr. 2, 2015.

(51) Int. Cl. A61K 8/60; A61Q 5/06; A61K 8/19

(52) U.S. Cl. CPC A61K 8/60; A61K 8/19; A61Q 5/06; A61K 2800/30; A61K 2800/594; A61K 2800/5922; A61K 2800/95

(57) ABSTRACT

This invention relates to a composition comprised of a sugar and a bicarbonate that in an aqueous solution acts as a long-lasting, non-synthetic fixative particularly suited for those with fine hair.

### FIELD OF INVENTION

This invention relates to the field of haircare. Specifically, the invention addresses hair care preparations with fixative properties used for hair styling and manageability without reliance on synthetic polymers. And more specifically, the invention addresses hair care preparations with fixative qualities that are especially appropriate for those with fine, limp hair.

### BACKGROUND

Fine hair is weaker than medium or coarse hair such that it easily becomes limp or flat even after styling.

It is known that individual strands of fine hair have less strength due to the significantly smaller diameter of each strand compared with coarser hair. Typically the diameter of a strand of fine hair is 70 microns. This is less than half the diameter of a strand of medium to course hair. Fine hair is also noted for having approximately half the internal protein of other hair types, also contributing to its lack of strength. As a result, fine hair can become limp or flat shortly after styling, even after using products such as volumizers, root lifters and thickeners.

While fine hair is typically a hereditary condition, the issues of fine hair are exacerbated with aging. During aging, hair is likely to undergo a miniaturization process, whereby the hair shaft decreases in diameter. In addition, during aging, hair follicles can become dormant (stop producing hair), causing hair to thin, further exacerbating the issues associated with fine, flat hair. After menopause many women complain about their fine hair and seek solutions to make their it look and feel fuller.

The vast majority of hair styling products, even those for fine hair, use synthetic polymers (e.g. acrylic and vinyl polymers and co-polymers) to help hair hold a style and create volume and fullness. However, as noted above, fine hair is easily weighed down by the typical hair care preparations made with these synthetic polymers, particularly when faced by common challenges such as brushing, compression (e.g. from hats, pillows), sweat/oil from the scalp, humidity, wind, and gravity.

When considering alternatives to the synthetic polymers commonly used in hair styling preparations, in addition to fixative effectiveness, it is desirable to focus on safety and sustainability. Use of safe and sustainable ingredients in haircare preparations is growing in importance with consumers, retailers (e.g. Whole Foods' Ingredient Standards), manufacturers, and regulators (e.g. California's increasing regulation of cosmetic ingredients).

Given these safety and sustainability considerations, saccharides are an appealing hair care ingredient. It is known that saccharides can form polymers. Additionally, the semi-crystalline and glass-like polymers created by saccharides and specifically disaccharides, tend to be ‘stiffer, harder and more dense’ than amorphous polymers (http://courses.chem.psu.edu/chem112/materials/polymers.html) making them an ideal fixative for fine limp hair. And saccharides, and specifically disaccharides, are water compatible such that their polymers are easily removed from hair with water alone or in combination with shampoo so they won't build-up and weigh fine hair down with multiple uses.

However, saccharides, including disaccharides, are also noted for their hydrophilic properties, inhibiting their use as as a styling fixative. Therefore, this invention seeks to address the hydrophilic qualities associated with saccharides in order to ensure fixative effectiveness for fine hair.

Of note, it is understood that polysaccharides and sugar alcohols have very different properties from monosaccharides and disaccharides.

### SUMMARY OF INVENTION

The present invention is directed to hair styling compositions which contain a saccharide or a mix of saccharides, preferably disaccharides, and bicarbonate, preferably sodium bicarbonate, in an aqueous or semi-aqueous solution that, when applied, coats the hair with a clear film that enhances hair flex strength and stiffness and is humidity resistant. Further, the resultant composition is a surprisingly lightweight, long-lasting fixative particularly suited to meet the unique needs of those with fine hair. And further, the compositions made according to this invention do not rely on synthetic polymers for fixative effectiveness.

### DETAILED DESCRIPTION

When discussing the concept of a hair ‘fixative’, we mean film forming polymers that are used to coat the hair with a thin layer of product to impart stiffness, flexible strength, and manageability to hair in order to create a lasting style (e.g. curl, waves or smoothness, lifting the ‘roots’ of the hair away from the scalp, creating the impression of volume, body and thickness). Products made with fixatives are applied to wet or dry hair and are styled with or without heat (e.g. hair dryers, curling irons, hot and steam rollers, non-heated roller, etc).

Beyond holding the hair in a desired shape after styling, qualities desired of fixatives include resilience, the ability to adhere to the hair without flaking, the ability to resist humidity, and the ability to impart a sheen or gloss to the hair. Further they should be easily removed by washing with water or shampoo. Products made with fixative polymers can be described as ‘hair sprays’, ‘root boosters’, ‘styling sprays’, ‘volumizers’, ‘setting lotions/creams’, ‘mousses’, ‘tonics’, leave-in styling sprays', and ‘gels’ among other terms. Further, beyond holding hair shape, products with fixative polymers may be marketed for hair protection from heat-styling, strengthening hair to prevent breakage and split-ends, thickening the hair, creating shine, and tinting or dying hair.

While typical hair care fixatives are made with synthetic polymers, this invention is concerned with the development of an composition that combines non-synthetic ingredients, leveraging the known crystalline, semi-crystalline and glass polymer properties of saccharides (e.g. sucrose, glucose, and fructose) combined with a bicarbonate (e.g. sodium bicarbonate) in an aqueous solution. It is believed that while heat styling, the composition according to this invention forms sugar polymers while the bicarbonate releases carbon dioxide infusing the sugar polymers with a honeycomb of tiny bubbles, thus increasing the stiffness of the sugar polymers and making them more resistant to the stickiness that might be expected of a fixative using sugar alone.

The composition according to this invention, coated on fine hair, has surprising and unexpected abilities to help fine hair retain it's shape when flexed by mechanical stressors such as brushing, compression and gravity, and when exposed to sweat or environmental humidity. Further, the composition described in this invention easily washes out of hair without the build-up expected after the use of many haircare preparations.

Method for Creating

A preparation according to this invention is a composition comprised of a sugar or sugar(s)—in this case a disaccharide, and a bicarbonate—in this case sodium bicarbonate, in an aqueous solution. Particularly disaccharide(s) are 15 percent by weight, but may vary from 4 percent by weight to 50 percent by weight of the preparation, and particularly sodium bicarbonate is 4 percent by weight but may vary from 0.5 percent to 15 percent by weight of the preparation.

This preparation according to invention may further be mixed with other ingredients such as water, aloe, vitamins, proteins, conditioners, perfumes, preservatives, stabilizers and emulsifiers to enhance formula performance, appeal and stability.

The said preparation described will have a pH level ranging from 4 to 9 and ideally 6.5 to 8.5. Of note, use of sodium bicarbonate in a preparation according to invention increases the pH level of the preparation. For fine hair, a slightly higher pH level (e.g. 7 to 8) is beneficial. Lower pH solutions (e.g. under 6) cause the hair cuticle to bind more tightly to the hair shaft which is ideal for those with coarser hair who seek to smooth their hair. But, for those with fine hair, causing the cuticle to bind tightly to the shaft of the hair further reduces hair diameter and thus the perception of hair thickness and volume. Using sodium bicarbonate in the preparation according to this invention creates a pH more akin to tap water (e.g. 7 to 8), causing the cuticle of fine hair to lift slightly further enhancing the appearance of hair thickness.

Using a spray pump device, a preparation containing composition according to this invention is sprayed on wet or damp hair after shampooing and conditioning. While blow drying hair, water evaporates from this preparation starting polymerization of the sugar(s). As evaporation continues, the concentration of sugar(s) increases, creating crystallization of the sugar polymers. Additionally heat generated by use of a hair dryer causes the sodium bicarbonate to release carbon dioxide bubbles, injecting the sugar polymers with minute air bubbles which act to stiffen these sugar polymer structures, resulting in surprising fixative hold, flex strength, shine, and resistance to humidity over time.

### PREFERRED EMBODIMENT OF INVENTION

The preferred preparation according to this invention is comprised of disaccharides and sodium bicarbonate in an aqueous solution plus aloe vera, natural extracts of herbs or leaves such as rosemary or ginseng, antioxidants, witch hazel and proteins such as silk protein, glycerine, and seed extracts such as grapefruit as well as ingredients to preserve and stabilize the preparation.

Usage

Using a spray pump container, agent can be applied to wet or dry hair and air dried or heat dried. However, it is noted that the best results are achieved when a preparation according to this invention is evenly applied to freshly washed and conditioned hair that has been towel dried, and then heat styled with a hair dryer and/or other styling devices. Further, volume can be increased by using implements (e.g. round brushes and styling devices) which lift the hair from the scalp as it's heat dried and allowing hair to cool while holding hair in the desired shape. Further it is noted that a preparation made according to this invention can be used to style hair with rollers or with curling irons.

As an example of usage, after washing and conditioning hair, towel dry and then evenly spray hair with a preparation made according to invention. with additional concentration on the roots of the hair. Gently comb or brush to distribute the preparation to the ends of the hair. Then heat-activate while styling with a round brush and blow dryer. While drying hair, allowing each section of hair to cool on a round brush to further stiffen the sugar polymers in order to maximize curl retention, volume and fullness.

For additional volume, product can be applied or reapplied to wet or dry hair in targeted sections and styled with a round brush. For example, taking a section of hair at the crown of the head, spray roots, brush hair to distribute the solution with agent to the ends of the hair, wrap hair around the round brush, and blow dry, while lifting hair from the scalp. Cool each section of hair before proceeding to the next section of hair. Finish by gently brushing or finger combing hair into the desired style.

It is also possible to use a hair preparation according to invention as a setting lotion to use with curlers (with or without heat) or even as an instant or dry shampoo to create the appearance of fuller, healthier hair between washes.

Supporting Data

A preparation according to this invention was tested for fixative effectiveness over time and ability to maintain fixative effectiveness in humid conditions.

Tests included the ‘ideal formula’ as described in the PREFERRED EMBODIMENT OF INVENTION as well as modifications to the ‘ideal formula’ in order to assess how these modifications impacted results.

To help ensure test reliability, the data was collected using the same hair sample (e.g. only one hair sample was used for all data). This hair sample was 24 cm long and was comprised of ‘fine’ hair’.

The Fixative Assessment

Fixative data was collected on six formulations:
1. Ideal Formula (includes both disaccharides and sodium bicarbonate)
2. Ideal Formula without disaccharides and without sodium bicarbonate
3. Ideal formula without sodium bicarbonate
4. Ideal formula without disaccharides
5. Ideal formula substituting disaccharides with glycerin (a sugar alcohol commonly used in hair care formulations)
6. Ideal formula substituting disaccharides with Erythritol (a sugar alcohol used in hair care preparations)

In each test, the hair sample was washed and conditioned, and excess water was removed by blotting gently with a paper towel. Then one of the six formulas was applied with a pump sprayer (spraying three times at a distance of six inches) and the formula was distributed by running a fine tooth comb through the hair sample twice. Following this, the prepared hair sample was wound inside a circular form 15 cm in diameter and dried (with hot air at 120 degrees fahrenheit for 30 seconds). The hair sample was then allowed to cool in the circular form for 60 seconds and then removed from the form and the curl length was measured (cm from the top of the hair sample to the lowest hanging hair while falling or dangling freely). This measurement was repeated after 10 minutes and 10 brush strokes, and again after 12 hours and an additional 10 brush strokes in order to determine the degree to which the hair sample lost it's ‘curl formation’ based on times and mechanical challenges. (Of note, in experiments with the Ideal Formula without the mechanical challenges, curl length was virtually unchanged after 12 hours. So the mechanical challenges to were added to better replicate real life conditions).

Shorter lengths (cm) signify tighter curl formation and thus greater fixative hold, while longer measurements indicate that the hair sample lost curl and ‘relaxed’ back toward it original length.

Per this data, the ideal formula (disaccharides plus sodium bicarbonate in an aqueous solution) exhibited the greatest curl retention, or fixative effectiveness, for fine hair.

Results were as follows:

### Fixative Assessment

After washing and conditioning, the damp hair sample was sprayed 3× with one of the formulas below. The formula was distributed thru sample with a fine comb and then blown dry for 30 seconds in a circle formation (diameter of 15 cm) After drying, the hair sample was allowed to cool for 60 sec and then measured while falling freely in order to determine the degree of curl retention. Data shows the curl length or distance from the top of the hair sample to the end of the hair sample after various time frames and mechanical challenges (i.e. brushing). Shorter distances equal greater curl retention or fixative benefits, longer distances equal less curl retention or fixative benefits. For reference, when dry and uncurled, the hair sample measured 24cm in length.

### Ideal Formula Comprises Disaccharides Plus Sodium Bicarbonate (i.e. Agent) Combined with Distilled Water, Witch Hazel, Aloe, Vitamin E, Silk Protein, and Grape Seed Extract

| Fixative results given time and mechanical challenges | | | | | |
|---|---|---|---|---|---|
| | Curl length after 10 | | Curl length after | Qualitative | pH |
| Curl length 60 | brush strokes and | Curl length after | 10 brush strokes | assessment of shine | pH |
| sec after | 10 minutes after | 12 hours after | and 12 hours after | and texture versus | variation |
| removing from | removing from | removing from | removing from | original hair sample | of |
| circle form (cm) | circle form (cm) | circle form (cm) | circle form (cm) | (no formula) | formulas |

Length of Fine Hair Sample no Formula and No Curl: 24 cm

| | | | | | | |
|---|---|---|---|---|---|---|
| Ideal formula without | 11.2 | 15.9 | 17.6 | 17.6 | Hair is smoother & | 8 |
| modification (i.e. with | | | | | shinier | |
| both disaccharides and | | | | | | |
| sodium bicarbonate) | | | | | | |
| Ideal formula minus | 18.5 | 18.5 | 19.2 | 19.2 | No change in hair | 6 |
| disacchrides and minus | | | | | texture or shine | |
| sodium bicarbonate | | | | | | |
| Ideal formula minus | 12.6 | 17.6 | 17.6 | 18.6 | Hair is smoother and | 6 |
| sodium bicarbonate | | | | | shinier | |
| Ideal formula minus | 18.5 | 18.5 | 19.1 | 19.2 | No noticable change in | 8 |
| disaccharides | | | | | hair texture or shine | |
| Ideal formula | 17.0 | 18.1 | 19.4 | 19.4 | Hair is smoother and | 8 |
| substituting | | | | | shinier, very easy to | |
| disaccharides with | | | | | brush | |
| glycerin | | | | | | |
| Ideal formula | 17.7 | 18.6 | 19.6 | 19.6 | After brushing, hair | 8 |
| substituting | | | | | looks dry and kinky, | |
| disaccharides with | | | | | hard to brush, lots of | |
| Erythritol (a sugar | | | | | static | |
| alcohol) | | | | | | |
| Commercial styling | 14.1 | 20.0 | 21.0 | 22.4 | After brushing, hair | 5 |
| spray w/fixative | | | | | looks kinky, hard to | |
| ingredients VP/VPA | | | | | brush | |
| Commercial styling | 11.3 | 17.0 | 18.5 | 19.1 | After brushing, hair | 6 |
| mousse w/fixative | | | | | looks dry | |
| ingredients VPA/VP | | | | | | |

The Humidity Assessment

Next we looked at fixative effectiveness in extremely humid conditions.

We reviewed two formulas, the ideal formula (including both disaccharides plus sodium bicarbonate), and the ideal formula without sodium bicarbonate.

For this challenge, we started with a temperature of 75 degrees fahrenheit and a relative humidity of 20%. At this temperature/humidity level, we applied the same methodology as described in the fixative test to prepare and measure the hair sample (wash/condition, apply formula, distribute evenly through the hair sample, place in a curl form, dry with hot air, allow the hair to cool for 60 seconds, then measure the curl length or distance from the top of the sample to the ends of the hair as the curled hair sample falls or dangles freely).

After the first measurement of the hair prepared as described above, the hair sample was subjected to increases in temperature and humidity. Over an elapsed time of six minutes, the relative humidity was raised from 20% to 55%, and concurrently the temperature was raised from 75 degrees fahrenheit to 85 degrees fahrenheit. When this temperature and humidity level was reached (again, in an elapsed time of 6 minutes), the length of the hair sample was measured again. The following data shows the change in the hair sample length from the start of the humidity challenge to the end of the humidity challenge.

The data reveals that a formula with both disaccharides and sodium bicarbonate exhibited improved humidity resistance (curl retention) compared to the formulas with disaccharides alone.

See data below.

### Fixative Assessment with Humidity

After washing and conditioning, the damp hair sample was sprayed 3× with one of the formulas below. The formula was distributed thru sample with a fine comb and then dried for 30 seconds in a circle formation (diameter of 15 cm) After drying, the hair sample was allowed to cool for 60 sec and then measured while fall freely in order to determine the degree of curl retention. Data shows the curl length or distance from the top of the curled hair sample to the end of the hair sample before and after changes in temperature and humidity. Shorter distances equal greater curl retention or fixative benefits, longer distances equal less curl retention or fixative effectiveness. (Of note, the original length of the dry hair sample with no formula and no curl is 24 cm)

### Ideal Formula Comprises Disaccharides Plus Sodium Bicarbonate (i.e. Agent) and Distilled Water, Witch Hazel, Aloe, Vitamin E, Silk Protein, and Grape Seed Extract

| | Fixative results under humid conditions | | |
|---|---|---|---|
| | | Curl length after increasing | |
| | Curl length 60 sec after | temperature to 85 F. and humidity | |
| | removing from the circle form | to 55% (Approximately 6 min of | pH assessment |
| | Temperature 75 F., | elapsed time and 7 minutes after | pH variation of |
| | humidity 20% (cm) | removing from the circle form (cm) | formulas |
| Ideal formula without modification | 11.3 | 15.1 | 8 |
| (i.e. including disaccharide plus | | | |
| sodium bicarbonate) | | | |
| Ideal formula minus sodium | 12.6 | 17.7 | 6 |
| bicarbonate (sugar version) | | | |

1. A non-permanent hair styling composition comprising;
a. at least one disaccharide or monosaccharide;
b. at least one bicarbonate such as sodium bicarbonate; and
c. at least some water

2. A method for making a non-permanent hair styling composition, the method comprising combining;
a. at least one monosaccharide or disaccharide;
b. at least one bicarbonate such as sodium bicarbonate;
c. at least some water;
d. and, after application to hair, use of a hair styling device to heat the composition to above 35 degrees celsius.

3. The composition according to any one of claims 1, 6 and 7, in which the composition is substantially free of synthetic polymers.

4. The composition according to any one of claims 1, 6, and 7, wherein said composition is a hair spray, a mousse, a gel, a tonic, leave-in hair spray, a hair lotion, a hair cream, or a conditioner.

5. The composition according to any one of claims 1, 6, and 7, wherein said composition includes a mixture of both monosaccharides and disaccharides.

6. A non-permanent hair styling composition comprising;
a. 4 percent to 50 percent by weight of at least one disaccharide or monosaccharide;
b. 0.5 percent to 15 percent by weight of at least one bicarbonate such as sodium bicarbonate; and
c. water or a mixture containing water

7. A non-permanent hair styling composition comprising;
a. at least one disaccharide or monosaccharide;
b. at least one bicarbonate such as sodium bicarbonate;
c. wherein water or a mixture containing water is added
